(19) United States
(12) Patent Application Publication
(10) Pub. No.: US 2012/0076847 A1
(43) Pub. Date: Mar. 29, 2012

(54) MEDICAL INSTRUMENT AND METHOD OF MANUFACTURING THE SAME

(75) Inventors: Wei-Te CHEN, Changhua County (TW); Wei-Jen Shih, Kaohsiung County (TW); Wei-Ching Wang, Kaohsiung County (TW); Jin-Long Jou, Kaohsiung City (TW)

(73) Assignee: METAL INDUSTRIES RESEARCH&DEVELOPMENT CENTRE, Kaohsiung (TW)

(21) Appl. No.: 12/979,781

(22) Filed: Dec. 28, 2010

(30) Foreign Application Priority Data
Sep. 29, 2010 (TW) 099133003

(51) Int. Cl. A61K 9/00; A61K 33/24; A61K 33/42; A61P 31/04; A61P 43/00; B32B 15/04; B05D 3/02; A61K 33/38; A61K 38/18; B82Y 5/00

(52) U.S. Cl. 424/423; 424/618; 424/649; 424/602; 424/400; 514/8.8; 428/457; 427/2.1; 977/810; 977/915

(57) ABSTRACT

A medical instrument and a manufacturing method thereof are provided. The medical instrument includes a biomedical metal layer and a polymer film. The polymer film is a biodegradable polymer material. The manufacturing method includes the following steps: providing the biomedical metal layer, immersing the biomedical metal layer in a polymer solution, performing a baking process on the biomedical metal layer coated with a polymer film, forming the biomedical metal layer coated with the polymer film, taking out the biomedical metal layer coated with the polymer film to fabricate the medical instrument. The biodegradable polymer film and the biomedical metal layer are combined into the medical instrument, so that a physician performs a surgery more easily. In addition, decomposition time of the polymer film can be preset, so as to achieve efficacy of blocking soft tissue cells having a higher growth rate.

### CROSS-REFERENCE TO RELATED APPLICATION

This application claims the benefit of Taiwan Patent Application No. 099133003, filed on Sep. 29, 2010, which is hereby incorporated by reference for all purposes as if fully set forth herein.

### BACKGROUND OF THE INVENTION

1. Field of Invention

The present invention relates to a medical instrument having biomedical metal and a method of manufacturing the same, and more particularly to a medical instrument having a biodegradable polymer film and a method of manufacturing the same.

2. Related Art

In a case that a patient has an excessively thin and concave gum bone that a subsequent tooth implantation operation cannot be performed, a proper bone tissue regeneration process must be adopted to perform Guide Tissue Regeneration (GTR), and a tissue barrier film is utilized to block soft tissue cells having a higher growth rate, so as to prevent the soft tissue cells from invading, and provide a stable space environment, so hard bone cells (such as cementum and alveolar bone) growing more slowly get to proliferate, differentiate, and grow. A preset bone tissue regeneration guide object is filled at a defective site, so as to utilize bone proliferation characteristics to reinforce the bone defective site for achieving bone strength and condition conforming to requirement of further surgeries and achieving an effect of bone healing and tooth fixing. This technology can further be developed into Guided bone regeneration (GBR), so as to be applied for rebuilding of bone defection.

In the prior art, composition of a conventional guide object for bone tissue regeneration and each implementation action thereof are applied to demand for bone tissue regeneration of a large site. Mainly, a defective site of a gum or a bone or a coating tissue such as a gingiva or a muscle around a bone proliferation site is cut, then an osteogenic material (such as autogenous bone, synthetic bone or heterogeneous bone) is filled at the bone defective site or a site where a bone needs to proliferate in thickness, and additionally a tissue isolation film or a tissue isolation film having a reinforcement support (titanium mesh) is covered on the osteogenic material, and finally the cut coating tissue is stitched. After the wound is recovered, one more surgery is required to take out the tissue isolation film.

Furthermore, an absorbable tissue isolation film without being taken out is also developed. However, for the composition and the implementation manner, because the osteogenic material is separated from the tissue isolation film (or the tissue isolation film having the reinforcement support), respective further surgeries are required resulting in operation inconveniences and also difficulties for the osteogenic material to be combined with the tissue isolation film (or the tissue isolation film having the reinforcement support), and particularly the absorbable tissue isolation film has low mechanical strength, and a material of the tissue isolation film is soft and weak, so it usually becomes more difficult for a physician to give the surgery. Additionally, most of tissue isolation films having the reinforcement support are usually incompatible with the coating tissue such as the gingiva or the muscle, so first the osteogenic material has to get combined with the peripheral gum or bone, the physician has to cut the coating tissue such as the gingiva or the muscle again, so as to take out the tissue isolation film having the reinforcement support. Therefore, the multiple surgeries further bring pain to the patient, increase infection opportunities, and increase surgery risks and cost.

### SUMMARY OF THE INVENTION

In view of this, in order to solve the problem, the present invention is directed to a medical instrument having biomedical metal and a method of manufacturing the same. For this medical instrument having the biomedical metal and the method of manufacturing the same, a biodegradable polymer film and a biomedical metal layer are combined into the medical instrument according to the present invention.

The present invention provides a medical instrument which comprises a biomedical metal layer and a polymer film. The polymer film is made of a biodegradable polymer material, which can be adjusted according to demands, so as to achieve the requirement of blocking soft tissue cells having a higher growth rate for more than three months.

Wherein, a material of the biomedical metal layer is titanium-based metal, titanium metal, titanium-containing alloy, cobalt-chromium-molybdenum alloy or stainless steel metal.

Wherein, the polymer film is formed on a second surface of the biomedical metal layer, and the second surface is opposite to the first surface.

Wherein, a shape of the biomedical metal layer of the medical instrument is defined through metal machining, and a method of the metal machining is laser pattern machining, electrochemical machining, acid etching machining or alkaline etching machining.

Wherein, the polymer film of the medical instrument is chitosan, collagen or gelatin.

Wherein, the polymer film of the medical instrument is added with an additive promoting tissue growth, promoting tissue healing or an antibacterial therapeutic effect, and the additive is nano gold, nano silver, calcium phosphate or bone morphogenetic protein (BMP).

Wherein, the medical instrument is an object implanted in a body or temporarily implanted in the body.

The present invention provides a method of manufacturing the medical instrument, which comprises: providing a biomedical metal layer, placing the biomedical metal layer in a holding container, injecting polymer solution into the holding container, forming a polymer film on the biomedical metal layer through a first baking process wherein the biomedical metal layer has a first surface coated with the polymer film, taking out the biomedical metal layer coated with the polymer film from the holding container, immersing it in a crosslinking agent solution to perform a crosslinking reaction within a predetermined time, and taking out the biomedical metal layer coated with the polymer film to perform a second baking process after cleaning the biomedical metal layer coated with the polymer film, so as to fabricate the medical instrument.

Wherein, a material of the biomedical metal layer is made of titanium-based metal, titanium metal, titanium-containing alloy, cobalt-chromium-molybdenum alloy or stainless steel metal.

Wherein, a shape of the biomedical metal layer is defined through a metal machining process, and the metal machining process is a laser pattern machining process, an electrochemical machining process, an acid etching machining process or an alkaline etching machining process.

Wherein, the polymer solution includes a biodegradable polymer material.

Wherein, the polymer solution is made of chitosan, collagen or gelatin.

Wherein, the polymer solution is added with an additive promoting tissue growth, promoting tissue healing or having an antibacterial therapeutic effect, and the additive is nano gold, nano silver, calcium phosphate or bone morphogenetic protein (BMP).

Wherein, the crosslinking agent is NaOH, short-chain polylactic acid, glutaraldehyde or pentylene glycol.

The present invention is characterized in that the biodegradable polymer film and the biomedical metal layer are combined into the medical instrument of the present invention, so that a physician performs a surgery more easily. In addition, decomposition time of the polymer film can be preset, so as to achieve efficacy of blocking soft tissue cells having a higher growth rate, avoid risks of an additional surgery for taking out, reduce patient pains, reduce infection opportunities, and decrease surgery risks and cost.

### BRIEF DESCRIPTION OF THE DRAWINGS

The present invention will become more fully understood from the detailed description given herein below for illustration only, and thus are not limitative of the present invention, and wherein:

FIG. 1 is a schematic sectional view of a structure of a medical instrument according to an embodiment of the present invention;

FIG. 2 is a schematic sectional view of a structure of a medical instrument according to another embodiment of the present invention;

FIG. 3 is a flow chart of a manufacturing method of a medical instrument 10 according to the present invention;

FIG. 4 shows a thin film degradation result of dissolving an experimental end product in acetic acid in a period of 0 to 35 days;

FIG. 5 shows a thin film degradation result of dissolving an experimental end product in acetic acid in a period of 35 to 80 days;

FIG. 6a is a picture after co-culture with cells without using the medical instrument of the present invention;

FIG. 6b is a picture after co-culture with cells using the medical instrument of the present invention;

FIG. 7a is a picture of a wound of an experimental animal before embedding the medical instrument of the present invention;

FIG. 7b is a picture of the wound of the experimental animal after embedding the medical instrument of the present invention;

FIG. 8a is a drawing before a medical instrument of the present invention is mounted;

FIG. 8b a drawing during when the medical instrument of the present invention is being mounted; and

FIG. 8c is a drawing after the medical instrument of the present invention is mounted.

### DETAILED DESCRIPTION OF THE INVENTION

Preferred embodiments of the present invention are illustrated in cooperation with the drawings as follows.

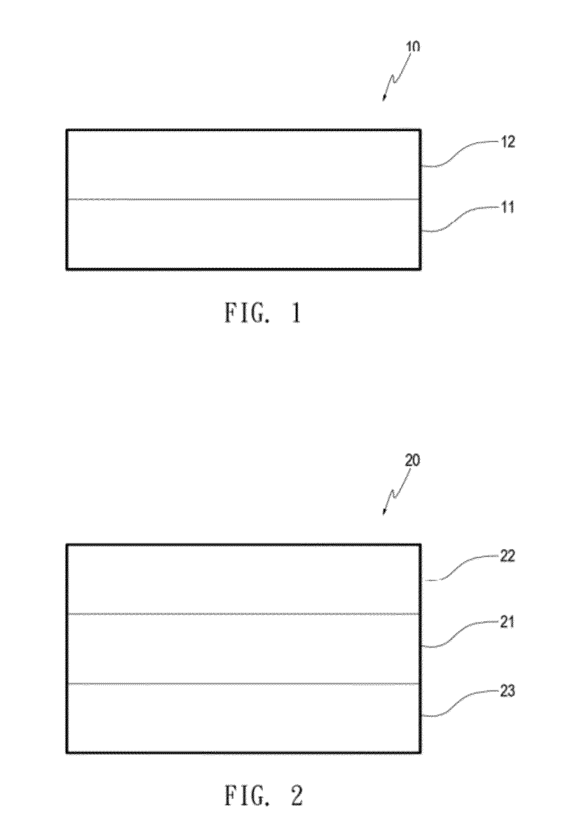

FIG. 1 is a schematic sectional view of a structure of a medical instrument according to an embodiment of the present invention. The medical instrument 10 according to the present invention at least includes a biomedical metal layer 11 and a polymer film 12 formed on the biomedical metal layer 11. The polymer film 12 is made of a biodegradable polymer material. The biomedical metal layer 11 is made of titanium-based metal, titanium metal, titanium-containing alloy, cobalt-chromium-molybdenum alloy or stainless steel metal. A shape of the biomedical metal layer 11 can be defined through a metal machining process, and the metal machining process can be a laser pattern machining process, an electrochemical machining process, an acid etching machining process or an alkaline etching machining process. The polymer film 12 can be made of a biodegradable polymer material, and the polymer film 12 can be made of chitosan, collagen or gelatin. The polymer film 12 can be added with an additive promoting tissue growth, promoting tissue healing or having an antibacterial therapeutic effect. The polymer film 12 can be added with nano gold, nano silver, calcium phosphate or bone morphogenetic protein (BMP). The medical instrument 10 is an object implanted in a body or an object temporarily implanted in the body.

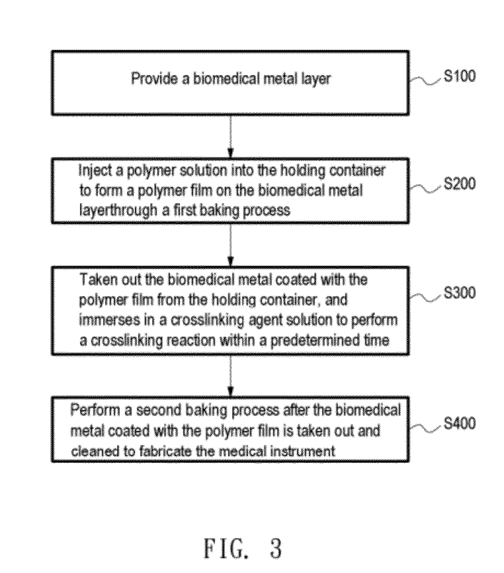

FIG. 2 is a schematic sectional view of a structure of a medical instrument 20 according to another embodiment of the present invention. The present invention medical instrument 20 at least includes a biomedical metal layer 21, a first polymer film 22, and a second polymer film 23, which are formed on two sides of the biomedical metal layer 21 respectively. The first polymer film 22 and the second polymer film 23 are made of a biodegradable polymer material. The biomedical metal layer 21 is made of titanium-based metal, titanium metal, titanium-containing alloy, cobalt-chromium-molybdenum alloy or stainless steel metal. A shape of the biomedical metal layer 21 can be defined through a metal machining process, and the metal machining process can be a laser pattern machining process, an electrochemical machining process, an acid etching machining process or an alkaline etching machining process. The first polymer film 22 and the second polymer film 23 can be made of a biodegradable polymer material, and the first polymer film 22 and the second polymer film 23 can be made of chitosan, collagen or gelatin. The first polymer film 22 and the second polymer film 23 can be added with an additive promoting tissue growth, promoting tissue healing or having an antibacterial therapeutic effect, and the first polymer film 22 and the second polymer film 23 can be added with nano gold, nano silver, calcium phosphate or BMP. The medical instrument 20 is an object implanted in a body or an object temporarily implanted in the body.

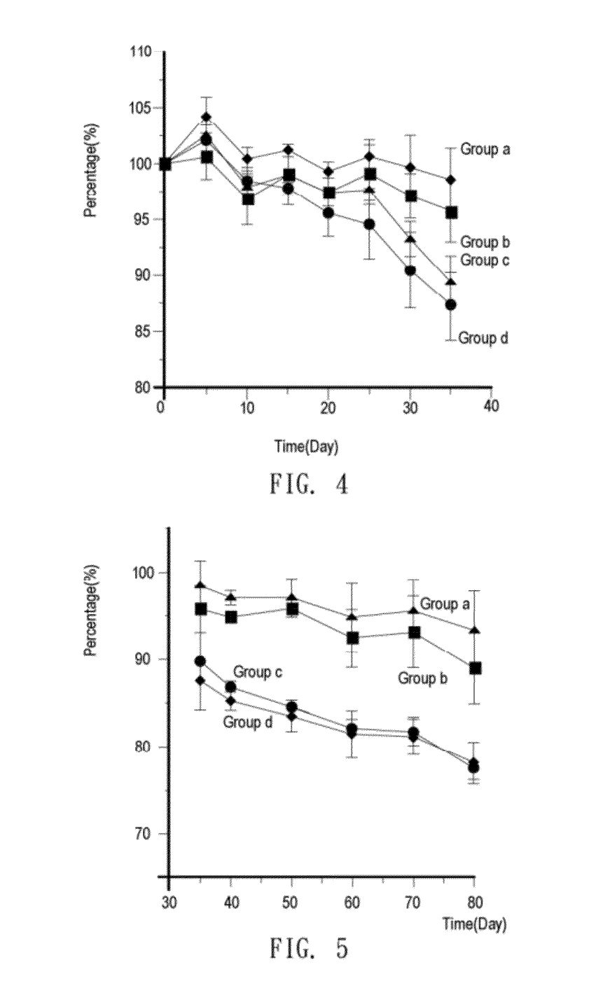

Referring to FIG. 1 and FIG. 3, FIG. 3 is a flow chart of a method of manufacturing the medical instrument 10 according to the present invention. The method of manufacturing the medical instrument 10 according to the present invention at least includes the following steps. A biomedical metal layer 11 is provided, and the biomedical metal layer 11 is placed in a holding container (Step S100). The biomedical metal layer 11 is horizontally placed in the holding container. The biomedical metal layer 11 is made of titanium-based metal, titanium metal, titanium-containing alloy, cobalt-chromium-molybdenum alloy or stainless steel metal. A shape of the biomedical metal layer 11 can be defined through a metal machining process. The metal machining process can be a laser pattern machining process, an electrochemical machining process, an acid etching machining process or an alkaline etching machining process. An inner wall of the holding container is a non-stick surface.

A polymer solution is injected into the holding container to form a polymer film 12 on a surface (e.g. lower surface) of the biomedical metal layer 11 through a first baking process (Step S200). A liquid level of the polymer solution contacts the surface of the biomedical metal layer 11. Since the inner wall of the holding container has the non-stick surface, when the polymer solution is baked through the first baking process, the polymer solution gradually dries and adheres to the lower surface of the biomedical metal layer 11, so as to form the biomedical metal layer 11 coated with the polymer film 12 at a single surface. The polymer solution can include a biodegradable polymer material. The polymer solution can include chitosan, collagen or gelatin. The polymer solution can be added with an additive promoting tissue growth, promoting tissue healing or having an antibacterial therapeutic effect. The polymer solution can be added with nano gold, nano silver, calcium phosphate or a BMP additive.

The biomedical metal layer 11 coated with the polymer film 12 is taken out from the holding container, and is immersed in a crosslinking agent solution to perform a crosslinking reaction within a predetermined time (Step S300). The crosslinking agent can be NaOH, short-chain polylactic acid, glutaraldehyde or pentylene glycol.

After the biomedical metal layer 11 coated with the polymer film 12 is taken out and cleaned, a second baking process is performed to fabricate the medical instrument 10 (Step S400). The medical instrument 10 can be an object implanted in a body or an object temporarily implanted in the body.

Referring to FIG. 2 and FIG. 3, FIG. 3 is a flow chart of a method of manufacturing the medical instrument 20 according to another embodiment of the present invention. The method of manufacturing the medical instrument 20 according to the present invention at least includes the following steps. A biomedical metal layer 21 is provided, and the biomedical metal layer 21 is placed in a holding container (Step S100). The biomedical metal layer 21 is made of titanium-based metal, titanium metal, titanium-containing alloy, cobalt-chromium-molybdenum alloy or stainless steel metal. A shape of the biomedical metal layer 21 can be defined through a metal machining process. The metal machining can be a laser pattern machining process, an electrochemical machining process, an acid etching machining process or an alkaline etching machining process. An inner wall of the holding container has a non-stick surface.

A polymer solution is injected into the holding container to form a polymer film 22, 23 on the first and second surfaces (e.g. upper and lower surfaces) of biomedical metal layer 21 through a first baking process (Step S200), the biomedical metal layer 21 is immersed in the polymer solution, and the polymer solution is in contact with the first and second surfaces of the biomedical metal layer 21. Since the inner wall of the holding container has the non-stick surface, when the polymer solution is baked through the first baking process, the polymer solution gradually dries and adheres to the surface of the biomedical metal layer 21, so as to form the biomedical metal layer 21 coated with the polymer films 22, 23 at double surfaces (i.e the first and second surfaces). The polymer solution can include a biodegradable polymer material. The polymer solution can be made of chitosan, collagen or gelatin. The polymer solution can be added with an additive promoting tissue growth, healing or an antibacterial therapeutic effect. The polymer solution can be added with nano gold, nano silver, calcium phosphate or a BMP additive.

The biomedical metal layer 21 coated with the polymer film 22, 23 is taken out from the holding container and immersed in a crosslinking agent solution, so as to perform a crosslinking reaction within a predetermined time (Step S300). The crosslinking agent can be NaOH, short-chain polylactic acid, glutaraldehyde or pentylene glycol.

After the biomedical metal layer 21 coated with the polymer film is taken out and cleaned, a second baking process is performed to fabricate the medical instrument 20 (Step S400). The medical instrument 20 can be an object implanted in a body or an object temporarily implanted in the body.

The present invention is illustrated hereinafter with reference to a first experiment example to a fifth experiment example, but the present invention is not merely limited to the following experiment examples.

### First Experiment Example

The first experiment example is a manufacturing method of a medical instrument according to the present invention, which at least includes the following steps.

A biomedical metal layer is provided. The biomedical metal layer adopted in this experiment example is made of titanium metal, and a required shape of the biomedical metal layer is defined through laser pattern machining. The biomedical metal layer is placed in a holding container.

A chitosan solution from 1 to 4 wt % is injected into the holding container, and then the holding container is placed in an oven at about 38 to 42 Celsius degrees for drying, so as to form a biomedical metal layer coated with the polymer film through a first baking process for about 22 to 26 hours. The chitosan solution is added with nano silver, calcium phosphate, and BMP.

The biomedical metal layer coated with the polymer film is taken out from the holding container, immersed in a 1N of NaOH (crosslinking agent) solution, and stands for about 0.5 to 4 hours at the room temperature, so that a full crosslinking reaction occurs between the chitosan solution and the 1N of NaOH (crosslinking agent), so as to strengthen mechanical strength of the coated polymer film through the crosslinking reaction.

Next, after being taken out and cleaned with deionized water, the biomedical metal layer coated with the polymer film is placed in a baking oven at about 38 to 42 Celsius degrees, and a second baking process is performed for about 22 to 24 hours, so as to finally fabricate the medical instrument of the present invention.

### Second Experiment Example

The second experiment example is to perform degradability test. By adjusting the content of chitosan, chitosan solutions having different concentrations are prepared, and it is analyzed and verified whether a chitosan thin film conforms to a long-term blocking effect (which generally requires more than three months, so as to conform to a bone tissue growth time), and is equipped with thin film mechanical strength conforming to the requirement, so as to maintain efficacy of blocking a soft tissue space.

According to the concentrations, the chitosan solutions are divided into four groups:

Group a: 1 wt % chitosan solution

Group b: 2 wt % chitosan solution

Group c: 3 wt % chitosan solution

Group d: 4 wt % chitosan solution

According to the manufacturing method of the medical instrument of the present invention, experimental end products of Group a, Group b, Group c, and Group d are fabricated respectively.

The experimental end products of Group a, Group b, Group c, and Group d are placed in a simulated solution filled with body fluid respectively, so as to simulate a degradation environment of the experimental end products of Group a, Group b, Group c, and Group d in an organism.

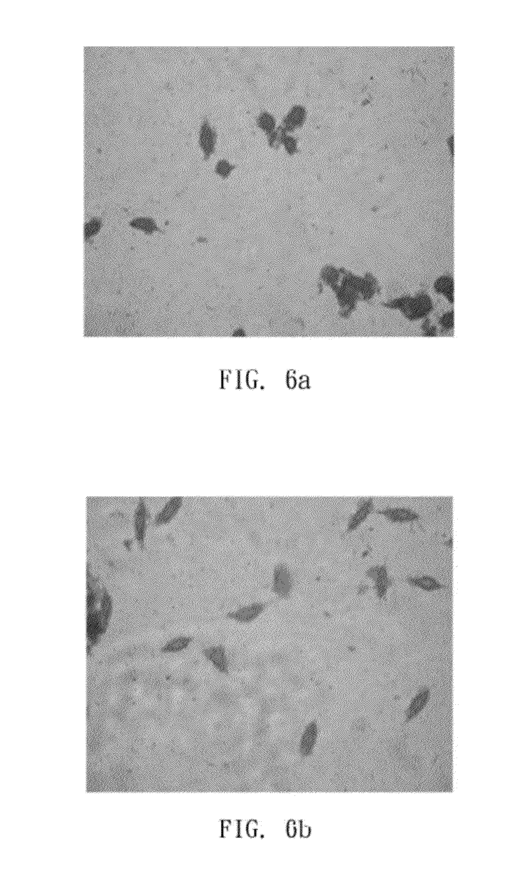

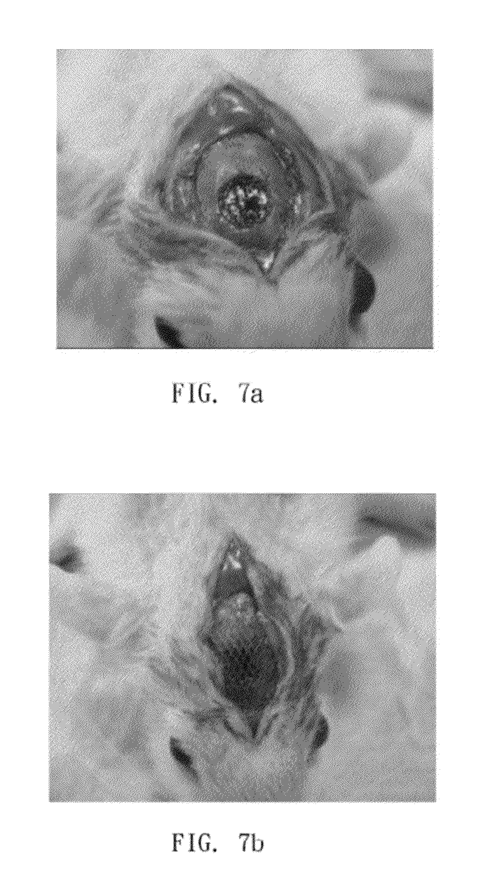

The experimental end products of Group a, Group b, Group c, and Group d are taken out from the simulated body fluid solution every 5 days for weighing, so as to obtain data such as a thin film degradation result of dissolving the experimental end products in the simulated body fluid in a period of 0 to 35 days as shown in FIG. 4 and a thin film degradation result of dissolving the experimental end products in the simulated body fluid in a period of 35 to 80 days as shown in FIG. 5.

It is proved from FIG. 4 and FIG. 5 that in the present invention, all the experimental end products of Group a, Group b, Group c, and Group d can actually keep more than 75% of the chitosan film in a period of 80 days, so the efficacy of blocking soft tissue cells having a higher growth rate can be achieved, and the degradation time of the chitosan film can be controlled through the concentrations of the chitosan solutions.

### Third Experiment Example

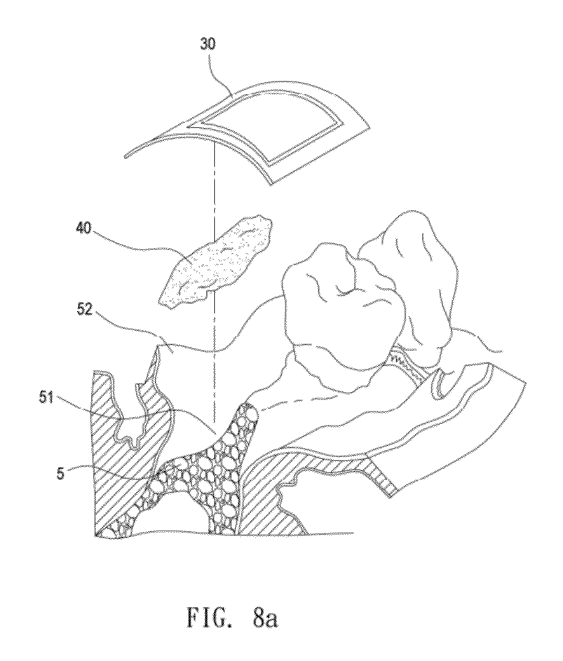

In the third experiment example, a cytotoxicity test is performed. FIG. 6a is a picture after co-culture with cells without using the medical instrument of the present invention and FIG. 6b is a picture after co-culture with cells using the medical instrument of the present invention. As can be seen from FIG. 6a and FIG. 6b, no matter whether the medical instrument of the present invention is used or not, a cell form thereof does not change.

Thus, a result of the cytotoxicity test shows that after co-culture of this medical instrument of the present invention with the cells, the cell form does not change, thus showing the cell compatibility of the medical instrument of the present invention.

### Fourth Experiment Example

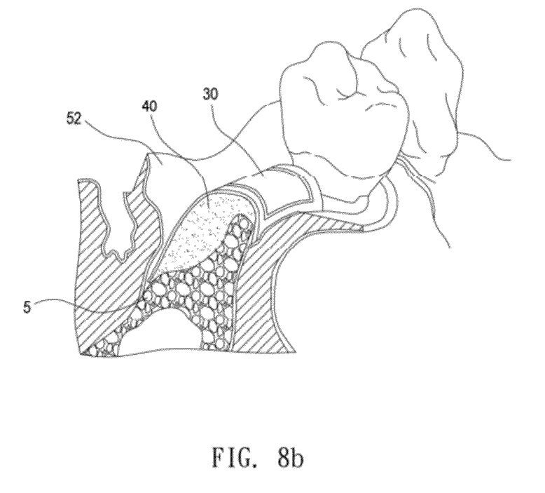

In the fourth experiment example, an animal experiment test is performed. FIG. 7a is a picture of a wound of an experimental animal before embedding the medical instrument of the present invention and FIG. 7b is a picture of the wound of the experimental animal after embedding the medical instrument of the present invention. As can be seen from FIG. 7a and FIG. 7b, the wound of the experimental animal is totally normal in appearance, and no inflammation phenomenon occurs, thus showing that the medical instrument of the present invention has good biological compatibility.

### Fifth Experiment Example

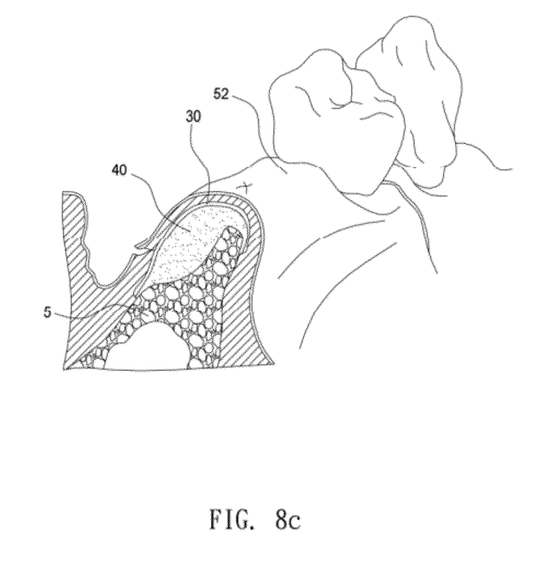

In the fifth experiment example, a process of mounting a medical instrument 30 of the present invention is illustrated. FIG. 8a is a drawing before the medical instrument 30 of the present invention is mounted; FIG. 8b is a drawing when the medical instrument 30 of the present invention is being mounted; and FIG. 8c is a drawing after the medical instrument 30 of the present invention is mounted. Mainly, a defective site 51 of a gum 5 (or bone) or a coating tissue 52 (such as gingiva or muscle) around a bone proliferation site is cut, then an osteogenic material 40 (such as autogenous bone, synthetic bone or heterogeneous bone) is filled at a bone defective site or a site where a bone needs to proliferate (thickness), the medical instrument 30 of the present invention is covered on the osteogenic material 40, and finally the cut coating tissue 52 is stitched. After a bone proliferation wound is recovered, the coating tissue 52 is cut to take out the medical instrument 30 of the present invention or a biodegradable polymer film begins to degrade and be absorbed by a human body as time passes. After being completely absorbed, the rest biomedical metal layer achieves the minimal area design according to different designed patterns, and by means of good biological compatibility of the biomedical metal, the processing of taking out can even be omitted.

In conclusion, according to the present invention, the medical instrument includes a biomedical metal layer and a polymer film having a biodegradable polymer material, and the composition of which can be adjusted according to the use demands, so as to control degradation time and achieve the requirement of blocking soft tissue cells having a higher growth rate for more than three months, and maintain good biological compatibility.

Although the present invention has been disclosed through the foregoing embodiments, they are not intended to limit the present invention. Equivalent replacements of variations and modifications made by persons skilled in the art without departing from the spirit and the scope of the present invention still fall within the protection scope of the present invention.

What is claimed is:

1. A medical instrument, comprising:
a biomedical metal layer; and
a polymer film, formed on a first surface of the biomedical metal layer, wherein the polymer film is made of a biodegradable polymer material.

2. The medical instrument according to claim 1, wherein the biomedical metal layer is made of titanium-based metal, titanium metal, titanium-containing alloy, cobalt-chromium-molybdenum alloy or stainless steel metal.

3. The medical instrument according to claim 1, wherein the polymer film is formed on a second surface of the biomedical metal layer, and the second surface is opposite to the first surface.

4. The medical instrument according to claim 1, wherein a shape of the biomedical metal layer of the medical instrument is defined through a metal machining process, and the metal machining process is a laser pattern machining process, an electrochemical machining process, an acid etching machining process or an alkaline etching machining process.

5. The medical instrument according to claim 1, wherein the polymer film of the medical instrument is made of chitosan, collagen or gelatin.

6. The medical instrument according to claim 1, wherein the polymer film of the medical instrument is added with an additive promoting tissue growth, promoting tissue healing or having an antibacterial therapeutic effect.

7. The medical instrument according to claim 1, wherein the additive is nano gold, nano silver, calcium phosphate or bone morphogenetic protein (BMP).

8. The medical instrument according to claim 1, wherein the medical instrument is an object implanted in a body or temporarily implanted in the body.

9. A method of manufacturing a medical instrument, at least comprising:
providing a biomedical metal layer, and placing the biomedical metal layer in a holding container;
injecting a polymer solution into the holding container, and forming a polymer film on the biomedical metal layer through a first baking process, wherein the biomedical metal layer has a first surface coated with the polymer film;
taking out the biomedical metal layer coated with the polymer film from the holding container, and immersing the biomedical metal layer coated with the polymer film in a crosslinking agent solution, so as to perform a crosslinking reaction within a predetermined time; and
performing a second baking process after cleaning the biomedical metal layer coated with the polymer film, so as to fabricate the medical instrument.

10. The method according to claim 9, wherein the biomedical metal layer is made of titanium-based metal, titanium metal, titanium-containing alloy, cobalt-chromium-molybdenum alloy or stainless steel metal.

11. The method according to claim 9, wherein a shape of the biomedical metal layer is defined through metal machining process, and a method of the metal machining process is a laser pattern machining process, an electrochemical machining process, acid an etching machining process or an alkaline etching machining process.

12. The method according to claim 9, wherein the polymer solution includes a biodegradable polymer material.

13. The method according to claim 9, wherein the polymer solution is made of chitosan, collagen or gelatin.

14. The method according to claim 9, wherein the polymer solution is added with an additive promoting tissue growth, promoting tissue healing or having an antibacterial therapeutic effect.

15. The method according to claim 14, wherein the additive is nano gold, nano silver, calcium phosphate or bone morphogenetic protein (BMP)

16. The method according to claim 9, wherein the crosslinking agent is NaOH, short-chain polylactic acid, glutaraldehyde or pentylene glycol.

17. The method according to claim 9, wherein the biomedical metal layer further has a second surface coated with the polymer film, and the second surface is opposite to the first surface.
